(19) United States
(12) Patent Application Publication
(10) Pub. No.: US 2020/0306263 A1
(43) Pub. Date: Oct. 1, 2020

(54) TOPICAL FORMULATION FOR INFLAMMATORY SKIN CONDITIONS

(71) Applicant: The Theraplex Company, LLC, Memphis, TN (US)

(72) Inventors: Peter Lio, Chicago, IL (US); Kevin Fowler, Memphis, TN (US); Michael Pietrangelo, Memphis, TN (US)

(21) Appl. No.: 16/365,870

(22) Filed: Mar. 27, 2019

(51) Int. Cl. A61K 31/573; A61K 36/899; A61K 36/889; A61K 31/202; A61K 47/06; A61K 9/00; A61P 37/00

(52) U.S. Cl. CPC A61K 31/573; A61K 36/899; A61K 36/889; A61P 37/00; A61K 47/06; A61K 9/0014; A61K 31/202

(57) ABSTRACT

The present disclosure provides a formulation for inflammatory skin conditions comprising about 0.05% to 10% by weight of colloidal oatmeal, about 5% about 30% by weight of coconut oil, about 0.25% to about 5% by weight of a corticosteroid, and a pharmaceutically acceptable carrier, emollient and/or diluent. The formulation further comprises in certain embodiments ferulic acid, petrolatum, a wax, and/or a silicone. The present disclosure further provides methods of treating, preventing or reducing the symptoms of inflammatory skin conditions, including eczema, psoriasis and seborrheic dermatitis, comprising topically administering to an affected area of skin a therapeutically effective amount of the formulations disclosed herein.

### BACKGROUND OF THE INVENTION

The present disclosure provides topical formulations for the treatment of inflammatory skin conditions and methods of treating inflammatory skin conditions.

Inflammatory skin diseases include chronic conditions such as eczema (atopic dermatitis), seborrheic dermatitis and psoriasis. Eczema in particular is characterized by red, patchy, or flaky skin. Additional symptoms include cracked or scaly skin, rawness from scratching and small bumps. Eczema generally begins in pediatric patients and can persist into adulthood. While there is no known cure for eczema, the symptoms are generally managed by moisturizing the skin, avoiding triggers, avoiding hot water, and using only mild skin care products.

Severe cases can be treated by prescription drugs, including oral corticosteroids, topical corticosteroids (prescription or over the counter), calcineurin inhibitors, or the injectable biologic medication dupilumab. Such medications may have undesirable side effects and can be costly. Furthermore, prescription medications may not be suitable for pediatric patients, particularly infants.

In addition to the discomfort of eczema, patients also may experience stress and embarrassment, disrupted sleep, and depression.

There is a continued need for more effective therapies for relieving the symptoms of inflammatory skin conditions, particularly eczema.

### BRIEF SUMMARY OF THE INVENTION

The present disclosure relates to topical formulations and methods for treating, preventing or reducing the symptoms of an inflammatory skin condition. The formulations are particularly useful for treating, preventing or reducing the symptoms of conditions such as eczema, psoriasis, and seborrheic dermatitis.

In one embodiment, the present disclosure provides a topical formulation for treating, preventing or reducing the symptoms of an inflammatory skin condition, comprising colloidal oatmeal, coconut oil, a corticosteroid and a pharmaceutically acceptable carrier, emollient and/or diluent. In a more particular embodiment, the formulation comprises about 0.05% to 10% by weight of colloidal oatmeal, about 5% about 30% by weight of coconut oil, about 0.25% to about 5% by weight of a corticosteroid, and a pharmaceutically acceptable carrier, emollient and/or diluent.

The formula further comprises one or more pharmaceutically acceptable carriers, emollients and/or diluents. For example, in some embodiments, the formulation comprises about 25% to about 75% by weight of petrolatum.

In certain embodiments, the formulation further comprises an acidifier, such as ferulic acid.

The formulation may further comprise a lipid thickener selected from the group consisting of ozokerite wax, beeswax, natural wax, synthetic wax and mixtures thereof.

The formulation in some embodiments comprises about 10% to about 40% by weight of a silicone selected from the group consisting of dimethicone, cyclomethicone, cyclopentasiloxane, cyclohexasiloxane, dimethiconol, and mixtures thereof.

The formulation may advantageously be provided as an ointment, cream or lotion, which has advantageous spreading characteristics and absorbs effectively into the skin.

The present disclosure further provides a method for treating, preventing or reducing the symptoms of an inflammatory skin condition comprising topically administering to an affected site any of the above-described formulations. In particular embodiments, the inflammatory skin condition is selected from the group consisting of eczema, psoriasis, and seborrheic dermatitis, and more particularly, the skin condition is eczema.

### DETAILED DESCRIPTION OF THE INVENTION

The present disclosure provides topical formulations for the application to the skin. The formulations described herein are useful for treating, preventing or reducing the symptoms of inflammatory skin conditions, such as eczema, psoriasis, and seborrheic dermatitis. The present formulations advantageously treat, prevent or reduce symptoms including, but not limited to, redness, itching, rash, flakiness, patchiness and scaliness, which are often experienced with inflammatory skin conditions. The formulations advantageously relieve these unpleasant symptoms while also having an acceptable skin feel, moisturization, and appearance, and also avoid tackiness.

The formulations advantageously combine colloidal oatmeal, coconut oil and a topical corticosteroid. In some embodiments, the formulation comprises about 0.05% to 10% by weight of colloidal oatmeal, about 5% about 30% by weight of coconut oil, about 0.25% to about 5% by weight of a corticosteroid, and a pharmaceutically acceptable carrier, emollient and/or diluent.

Colloidal oatmeal (also often referred to as oat beta glucan) is a naturally sourced anti-irritating and skin-soothing ingredient. The formulation comprises in some embodiments about 0.05% to 10%, about 0.1% to about 5%, about 0.5 to about 2.5%, about 0.5 to about 1.5% by weight colloidal oatmeal. In a particular embodiment, the formulation comprises about 1% by weight of the colloidal oatmeal.

The formulation comprises coconut oil. Coconut oil is extracted from the meat of matures coconuts of the coconut palm Cocos nucifera. It contains high levels of saturated fats, the most prevalent being lauric acid, myristic acid and palmitic acid. In some embodiments, the formulation comprises about 5% to about 30% by weight of the coconut oil. In other embodiments, the formulation comprises about 5% to about 25%, about 10% to 25%, about 10 to about 20%, about 12 to about 18%, or about 15% by weight of the coconut oil.

While not being bound by any particular theory, it is believed that the coconut oil as incorporated in to the present formulations advantageously balances the skin microbiota in the affected area, thereby improving the activity of the colloidal oatmeal, corticosteroid and other ingredients present in the formulation. Thus, the ingredients as formulated are believed to provide an unexpected synergistic effect.

The formulation comprises a topical steroid in an amount ranging from about 0.01% to about 5% by weight of the formulation. In other embodiments, the topical steroid is present in an amount ranging from about 0.25% to about 2.5%, about 0.5% to about 2.5%, 0.5% to about 1%, or about 1% by weight of the formulation.

In some embodiments, the topical steroid is selected from the group consisting of hydrocortisone, fluocinolone, desonide, prednicarbate, fluticasone, mometasone, triamcinolone, flurandrenolide, betamethasone, amcinonide, halcinonide, fluocinonide, diflorasone, halbetasol, clobetasol, and pharmaceutically acceptable salts thereof. Particular corticosteroids include hydrocortisone, hydrocortisone butyrate, hydrocortisone probutyrate, hydrocortisone valerate, fluocinolone acetonide, desonide, prednicarbate, fluticasone propionate, mometasone furoate, triamcinolone acetonide, flurandrenolide, betamethasone diproprionate, amcinonide, halcinonide fluocinonide, diflorasone diacetate, halbetasol prop a and clobetasol proprionate.

In one embodiment, the topical steroid is hydrocortisone in an amount ranging from about 0.05% to about 5% by weight of the formulation. In other embodiments, the hydrocortisone is present in an amount of about 0.25% to about 2.5%, about 0.5% to about 2.5%, 0.5% to about 1%, or about 1% by weight of the formulation.

In some embodiments, the formulation further comprises an acidifier, which may serve as an antioxidant, such as ferulic acid, lactic acids and other stabilized acids. In certain embodiments, the formulation comprises about 0.1 to about 10%, about 0.5 to about 5%, about 0.5% to about 2.5%, or about 1% by weight of an acidifier. In a particular embodiment, the acidifier is ferulic acid.

In certain embodiments, the formulation comprises about 25% to about 75% by weight of a pharmaceutically acceptable carrier, emollient and/or diluent. The carrier can be petrolatum, such as petrolatum Merkur 773 available from Sasol Performance Chemicals. In certain embodiments, the formulation comprises about 25% to about 75%, about 30% to about 70%, about 35% to about 65%, about 40% to about 60%, about 45% to about 55%, or about 50% by weight of petrolatum.

The present formulations may further comprise about 1% to about 15% by weight of a lipid thickener. In the embodiments, the formulation comprises about 2% to about 10%, about 5% to about 9% or about 7% by weight of the lipid thickener. The lipid thickener is selected from the group consisting of ozokerite wax, beeswax, natural wax, synthetic wax and mixtures thereof. In certain embodiments, the formulation comprises about 1% to about 15%, about 2% to about 10%, about 5% to about 9% or about 7% by weight of ozokerite wax.

In some embodiments, the formulation further comprises a silicone. While not being bound theory, silicone ingredients are believed to allow the formulation to spread easily on the skin and to contact the cracks, fissures, and other poorly accessible crevices in the skin. Many siloxanes are also volatile and evaporate after coating the skin, thus reducing tackiness. Examples of siloxanes suitable for the present formulations include dimethicone, cyclomethicone, cyclopentasiloxane (cyclomethicone D5), cyclohexasiloxane (cyclomethicone D6), dimethiconol, and mixtures thereof.

Accordingly, the formulations comprise in some embodiments about 10% to about 40%, about 15% to about 35%, about 20% to about 30%, or about 25% by weight of a siloxane. In a particular embodiment, the siloxane is cyclopentasiloxane.

The formulation described here may be provided as an ointment, cream, or lotion. The formulation preferably has a viscosity that is suitable from easy handling and applying to the affected area of the skin. The formulation may further be packaged into a tube, squeeze bottle, pump bottle, ampoules, or any other suitable container.

In a particular embodiment, a topical formulation for treating, preventing or reducing an inflammatory skin condition, comprises about 0.05% to 10% by weight of colloidal oatmeal, about 5% about 30% by weight of coconut oil, about 0.25% to about 5% by weight of a corticosteroid, about 25% to about 75% by weight of petrolatum, about 0.1 to about 10% of ferulic acid, about 1% to about 15% by weight of ozokerite wax, and about 10% to about 40% by weight of cyclopentasiloxane.

In particular embodiments, the corticosteroid is hydrocortisone.

In another embodiment, the formulation comprises about 0.1% to about 5%, by weight of colloidal oatmeal, about 5% to about 25% by weight of coconut oil, about 0.25% to about 2.5% by weight of hydrocortisone, about 35% to about 65% by weight of petrolatum, about 0.5 to about 5%, of ferulic acid, about 2% to about 10% by weight of ozokerite wax, and about 15% to about 35% by weight of cyclopentasiloxane.

In another embodiment, the formulation comprises about 0.5 to about 2.5% by weight of colloidal oatmeal, about 10 to about 20% by weight of coconut oil, about 0.5% to about 2.5% by weight of hydrocortisone, about 40% to about 60% by weight of petrolatum, about 0.5% to about 2.5% of ferulic acid, about 4% to about 9% by weight of ozokerite wax, and about 20% to about 30% by weight of cyclopentasiloxane.

In another embodiment, the formulation comprises about 1% by weight of colloidal oatmeal, about 15% by weight of coconut oil, 1% by weight of hydrocortisone, about 50% by weight of petrolatum, about 1% of ferulic acid, about 7% by weight of ozokerite wax, and about 15% by weight of cyclopentasiloxane.

The present disclosure further provides a method of treating, preventing or reducing the symptoms of an inflammatory skin condition comprising topically administering to an affected site the formulation described above.

In certain embodiments, the formulation is topically administered to an affected site from about 1 to about 5 times per day. The inflammatory skin condition is selected from the group consisting of eczema, psoriasis, and seborrheic dermatitis. More particularly, the inflammatory skin condition is eczema.

The terms “topical administration” or “topically administered” describe the introduction of the formulation to a subject's skin, particularly on an area affected by an inflammatory skin condition. The administration may further include rubbing the formulation into the skin the further improve absorption.

The term “therapeutically effective amount” describes a quantity of the formulation to achieve a desired effect in a subject being treated with the formulation. For example, this can be the amount of the formulation necessary to reduce, alleviate and/or prevent itching, burning, flaking, or patchiness of the skin. In some embodiments, a “therapeutically effective amount” is sufficient to reduce, eliminate or prevent the symptoms of inflammatory skin conditions.

By “reducing”, as in reducing symptoms of an inflammatory skin condition, is meant a lessening of the severity or shortening the length of time a subject experiences symptoms.

In other embodiments, a “preventative” treatment is administered to a subject who does not exhibit signs of inflammatory skin conditions or exhibits only early signs for the purpose of decreasing the risk of developing more severe symptoms.

Upon application of the topical formulation, the components of the formulation penetrate the surface of the skin and the subject experiences a relief of symptoms, including itching, burning, redness, swelling, patchiness and flakiness. Suitably, the subject experiences at least a partial reduction in the intensity of at least one of these symptoms. Preferably, the subject experiences almost total alleviation of symptoms.

The topical formulation of the invention is available to a plurality of subjects. The term “subject” is used in its broadest sense. In a preferred embodiment, the subject is a mammal. More preferably, the subject is a human. Non-limiting examples of mammals include humans, dogs, cats, horses, cows, sheep, goats and pigs. Preferably, a subject includes any human or non-human mammal, including for example, a primate, cow, horse, pig, sheep, goat, dog, cat, or rodent, capable of being treated for an inflammatory skin condition.

The present formulations can comprise, consist of, or consist essentially of any of the required or optional ingredients and/or limitations described herein.

### EXAMPLES

Table 1 depicts an exemplary formulation in accordance with the present disclosure

TABLE 1

| | Eczema Therapy Ointment | | Batch size 500 g |
|---|---|---|---|
| | INGREDIENT | % | Amt. In Batch (g) |
| 1 | PHASE A | | |
| 2 | Petrolatum Merkur 773 (MCP) | 50.000 | 250.00 |
| 3 | Ozokerite Wax (MCP) | 7.000 | 35.00 |
| 4 | Oat Beta Glucan (MCP) | 1.000 | 5.00 |
| 5 | Coconut Oil (Natural Sourcing) | 15.000 | 75.00 |
| 6 | Ferulic Acid (Nature & Nurture) | 1.000 | 5.00 |
| | Hydrocortisone (Dastech) | 1.000 | 5.00 |
| 7 | PHASE B | | |
| | Dow Corning 245 Fluid (MCP) | 25.000 | 125.00 |
| | TOTAL | 100.000 | 500.00 |

Procedure: Add petrolatum to a suitable vessel and heat to 175-185 F with slow mixing. Add remaining Phase A ingredients except hydrocortisone with fast mixing. Cool to 120 F and add hydrocortisone with mixing. Cool to 110 F with mixing. Add Phase B to Phase A with mixing. When uniform, fill into suitable packaging and cool to room temp. The formulation produced is a white ointment.

Eczema Therapy: Several patients suffering from chronic eczema were treated with the formulation of table 1. The formulation was administered to the areas of skin affected by eczema and was well tolerated (e.g., non-irritating). Dramatic reductions in eczema symptoms were observed and repeated flare ups of chronic eczema were significantly reduced.

Thus, although there have been described particular embodiments of the present invention of a new and useful Eczema formula it is not intended that such references be construed as limitations upon the scope of this invention except as set forth in the following claims.

1. A topical formulation for treating, preventing or reducing an inflammatory skin condition, comprising:
about 0.05% to 10% by weight of colloidal oatmeal,
about 5% about 30% by weight of coconut oil,
about 0.25% to about 5% by weight of a corticosteroid, and
a pharmaceutically acceptable carrier, emollient and/or diluent.

2. The formulation of claim 1, wherein the formulation comprises about 0.05% to about 2.0% by weight of the colloidal oatmeal.

3. The formulation of claim 1, wherein the formulation comprises about 10% to about 20% by weight of the coconut oil.

4. The formulation of claim 1, wherein the formulation comprises about 0.5 to about 2.5% by weight of the corticosteroid.

5. The formulation of claim 1, wherein the corticosteroid is selected from the group consisting of hydrocortisone, fluocinolone, desonide, prednicarbate, fluticasone, mometasone, triamcinolone, flurandrenolide, betamethasone, amcinonide, halcinonide, fluocinonide, diflorasone, halbetasol, clobetasol, and pharmaceutically acceptable salts thereof.

6. The formulation of claim 5, wherein the corticosteroid is hydrocortisone.

7. The formulation of claim 1, wherein the formulation further comprises about 0.1 to about 10% of an acidifier.

8. The formulation of claim 7, wherein the acidifier is ferulic acid.

9. The formulation of claim 1, wherein the pharmaceutically acceptable carrier, emollient and/or diluent comprises about 25% to about 75% by weight of petrolatum.

10. The formulation of claim 9, wherein the formulation comprises about 35 to about 65% by weight of the petrolatum.

11. The formulation of claim 1, further comprising about 1% to about 15% by weight of a lipid thickener.

12. The formulation of claim 11, wherein the lipid thickener is selected from the group consisting of ozokerite wax, beeswax, natural wax, synthetic wax, and mixtures thereof.

13. The formulation of claim 12, wherein the lipid thickener is ozokerite wax.

14. The formulation of claim 1, further comprising about 10% to about 40% by weight of a silicone selected from the group consisting of dimethicone, cyclomethicone, cyclopentasiloxane, cyclohexasiloxane, dimethiconol, and mixtures thereof.

15. The formulation of claim 14, wherein the silicone is cyclopentasiloxane.

16. The formulation of claim 1, wherein the formulation is an ointment, cream, or lotion.

17. A method of treating, preventing or reducing the symptoms of an inflammatory skin condition comprising topically administering to an affected area of skin a therapeutically effective amount of the formulation of claim 1.

18. The method of claim 17, wherein the formulation is topically administered from about 1 to about 5 times per day.

19. The method of claim 17, wherein the inflammatory skin condition is selected from the group consisting of eczema, psoriasis, and seborrheic dermatitis.

20. The method of claim 19, wherein the inflammatory skin condition is eczema.
